(19) United States
(12) Patent Application Publication
(10) Pub. No.: US 2026/0166052 A1
(43) Pub. Date: Jun. 18, 2026

(54) Systems and Methods for Verifying Iboga Alkaloid Treatment using Temporospatial Connectivity

(71) Applicant: The Board of Trustees of the Leland Stanford Junior University, Stanford, CA (US)

(72) Inventors: Nolan R. Williams, Incline Village, NV (US); Anish Mitra, Stanford, CA (US)

(73) Assignee: The Board of Trustees of the Leland Stanford Junior University, Stanford, CA (US)

(21) Appl. No.: 19/127,014

(22) PCT Filed: Nov. 2, 2023
(86) PCT No.: PCT/US2023/078533

§ 371 Date: May 2, 2025

Related U.S. Application Data
(60) Provisional application No. 63/382,079, filed on Nov. 2, 2022.

(51) Int. Cl. A61K 31/55; A61K 45/06; G06T 7/00; G16H 20/70

(52) U.S. Cl. CPC A61K 31/55; A61K 45/06; G06T 7/0016; G16H 20/70; G06T 2207/10088; G06T 2207/30016

(57) ABSTRACT

Systems and methods for verifying iboga alkaloid treatment using temporospatial connectivity in accordance with embodiments of the invention are illustrated. One embodiment includes a treatment verification system, including a processor, and a memory containing a treatment verification application that configures the processor to obtain a pre-treatment functional imaging data of a patient's brain, identify pre-treatment disordered lagged correlations between different brain regions associated with a neuropsychiatric disorder using the functional imaging data, treat the patient with an iboga alkaloid or salt thereof in an amount to achieve a physiologic effect, obtain a post-treatment functional imaging data of the patient's brain, identify reversion of the pre-treatment disordered lagged correlations in the post-treatment functional imaging data, and provide a verification that treatment with the iboga alkaloid or salt thereof provided a therapeutic effect in treating the neuropsychiatric condition.

### CROSS-REFERENCE TO RELATED APPLICATIONS

The current application claims the benefit of and priority under 35 U.S.C. § 119(e) to U.S. Provisional Patent Application No. 63/382,079 entitled “Systems and Methods for Neurostimulation Targeting using Temporospatial Connectivity” filed Nov. 2, 2022. The disclosure of U.S. Provisional Patent Application No. 63/382,079 is hereby incorporated by reference in their entireties for all purposes.

### FIELD OF THE INVENTION

The present invention generally relates to the empirical identification of certain types of depression, generation of personalized neurostimulation targets based on temporospatial analysis of brain activation to treat said types of depression, and confirmation of effective treatment.

### BACKGROUND

A connectome is a comprehensive map of neural connections in the brain. Connectomics is a science that comprises of two broad fields, structural connectivity and functional connectivity. Structural connectivity considers the neuron to neuron connectivity in the brain and uses this information to define network architectures. In contrast, functional connectivity is built on communication between neurons and/or neuronal ensembles, and defines networks on the basis of mutual communication defined using metrics such as temporal correlation. A number of functional networks which are typically shared amongst all humans are known such as (but not limited to) the default mode network, the salience network, the attention network, any many more.

Brain activity can be non-invasively measured using any of a number of functional neuroimaging techniques including (but not limited to), positron emission tomography (PET), functional magnetic resonance imaging (fMRI), functional near-infrared spectroscopy (fNIRS), and functional ultrasound imaging (fUS).

### SUMMARY OF THE INVENTION

Systems and methods for verifying iboga alkaloid treatment using temporospatial connectivity in accordance with embodiments of the invention are illustrated. One embodiment includes a treatment verification system, including a processor, and a memory containing a treatment verification application that configures the processor to obtain a pre-treatment functional imaging data of a patient's brain, identify pre-treatment disordered lagged correlations between different brain regions associated with a neuropsychiatric disorder using the functional imaging data, treat the patient with an iboga alkaloid or salt thereof in an amount to achieve a physiologic effect, obtain a post-treatment functional imaging data of the patient's brain, identify reversion of the pre-treatment disordered lagged correlations in the post-treatment functional imaging data, and provide a verification that treatment with the iboga alkaloid or salt thereof provided a therapeutic effect in treating the neuropsychiatric condition.

In a further embodiment, the neuropsychiatric condition is major depressive disorder.

In still another embodiment, the neuropsychiatric condition is suicidal ideation.

In a still further embodiment, the iboga alkaloid or salt thereof is provided with a cardioprotective agent.

In yet another embodiment, to identify pre-treatment disordered lagged correlation, the treatment verification application further configures the processor to label voxels in the pre-treatment functional brain imaging data that make up a dorsolateral prefrontal cortex and an anterior cingulate cortex in the pre-treatment functional brain imaging data using a brain atlas, generate a whole-brain lagged correlation map using the voxels labelled as making up the anterior cingulate cortex as a reference region, where the whole-brain lagged correlation map includes a peak correlation magnitude and a peak temporal delay, compute a lag for all pairs of voxels containing grey matter, and assemble the computed lags into a time-delay matrix.

In a yet further embodiment, to identify pre-treatment disordered lagged correlations, the treatment verification application further configures the processor to compute the mean delay over all columns of the time-delay matrix to produce a lag projection map.

In another additional embodiment, to identify post-treatment disordered lagged correlation, the treatment verification application further configures the processor to label voxels in the post-treatment functional brain imaging data that make up a dorsolateral prefrontal cortex and an anterior cingulate cortex in the pre-treatment functional brain imaging data using a brain atlas, generate a whole-brain lagged correlation map using the voxels labelled as making up the anterior cingulate cortex as a reference region, where the whole-brain lagged correlation map includes a peak correlation magnitude and a peak temporal delay, compute a lag for all pairs of voxels containing grey matter, and assemble the computed lags into a time-delay matrix.

In a further additional embodiment, to identify post-treatment disordered lagged correlations, the treatment verification application further configures the processor to compute the mean delay over all columns of the time-delay matrix to produce a lag projection map.

In another embodiment again, pre-treatment functional imaging data and post-treatment functional imaging data are produced using a magnetic resonance imaging machine.

In a further embodiment again, the post-treatment functional imaging data is obtained between 1 day and 1 month post-treatment.

One embodiment includes a method of verifying treatment with iboga alkaloids, including obtaining a pre-treatment functional imaging data of a patient's brain, identifying pre-treatment disordered lagged correlations between different brain regions associated with a neuropsychiatric disorder using the functional imaging data, treating the patient with an iboga alkaloid or salt thereof in an amount to achieve a physiologic effect, obtaining a post-treatment functional imaging data of the patient's brain, identifying reversion of the pre-treatment disordered lagged correlations in the post-treatment functional imaging data, and providing a verification that treatment with the iboga alkaloid or salt thereof provided a therapeutic effect in treating the neuropsychiatric condition.

In still yet another embodiment, the neuropsychiatric condition is major depressive disorder.

In a still yet further embodiment, the neuropsychiatric condition is suicidal ideation.

In still another additional embodiment, the iboga alkaloid or salt thereof is provided with a cardioprotective agent.

In a still further additional embodiment, identifying pre-treatment disordered lagged correlation includes labeling voxels in the pre-treatment functional brain imaging data that make up a dorsolateral prefrontal cortex and an anterior cingulate cortex in the pre-treatment functional brain imaging data using a brain atlas, generating a whole-brain lagged correlation map using the voxels labelled as making up the anterior cingulate cortex as a reference region, where the whole-brain lagged correlation map includes a peak correlation magnitude and a peak temporal delay, computing a lag for all pairs of voxels containing grey matter, and assembling the computed lags into a time-delay matrix.

In still another embodiment again, to identify pre-treatment disordered lagged correlations, the treatment verification application further configures the processor to compute the mean delay over all columns of the time-delay matrix to produce a lag projection map.

In a still further embodiment again, identifying post-treatment disordered lagged correlation includes labeling voxels in the post-treatment functional brain imaging data that make up a dorsolateral prefrontal cortex and an anterior cingulate cortex in the pre-treatment functional brain imaging data using a brain atlas, generating a whole-brain lagged correlation map using the voxels labelled as making up the anterior cingulate cortex as a reference region, where the whole-brain lagged correlation map includes a peak correlation magnitude and a peak temporal delay, computing a lag for all pairs of voxels containing grey matter, and assembling the computed lags into a time-delay matrix.

In yet another additional embodiment, identifying post-treatment disordered lagged correlations further includes computing the mean delay over all columns of the time-delay matrix to produce a lag projection map.

In a yet further additional embodiment, pre-treatment functional imaging data and post-treatment functional imaging data are produced using a magnetic resonance imaging machine.

In yet another embodiment again, the post-treatment functional imaging data is obtained between 1 day and 1 month post-treatment.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 is a system diagram for a treatment verification system in accordance with an embodiment of the invention.

FIG. 2 is a block diagram for a treatment verifier in accordance with an embodiment of the invention.

FIG. 3 is a flow chart for a treatment verification in accordance with an embodiment of the invention.

FIG. 4 is a flow chart for a treatment verification process for computing activation lag in accordance with an embodiment of the invention.

FIG. 5A-D is a set of charts illustrating computation of functional lag and a target in accordance with an embodiment of the invention.

FIG. 6 is a series of brain images showing example differences in temporal changes in activation over time between high responders and low responders in accordance with an embodiment of the invention.

### DETAILED DESCRIPTION

Depression is a debilitating mental condition that occurs with relatively high frequency in the population. Many forms of clinical depression are the result of pathological brain circuitry. Multiple different brain circuitry abnormalities can give rise to depression. Studies have suggested that major depressive disorder (MDD) is likely caused by aberrant communication patterns in brain-wide networks. However, a mechanism-based biomarker for MDD has remained elusive. Traditionally, most studies have focused on either tonic activity levels in a given brain region or interactions between brain regions on the basis of zero-lag correlation magnitude (“functional connectivity”). The limitation of this conventional view of connectivity is that there is no assignment of signaling directionality. Thus, a circumstance in which brain region A signals to brain region B can appear identical to a circumstance in which the direction of communication is reversed, even though the neurobiological implications of communication direction may be profound.

Systems and methods described herein instead take a spatiotemporal view of connectivity, where lag times in activation between different correlated brain structures are measured in order to derive directionality of activation. For example, a particular type of depression is left dorsolateral prefrontal cortex (L-DLPFC)—anterior cingulate cortex (ACC) based major depressive disorder (MDD), where the depression arises from dysregulation of at least these two brain regions. In particular activation irregularly flows from the ACC to the DLPFC (i.e. ACC activation leads correlated DLPFC activation). Many other mental conditions may arise from similar irregular activation patterns systems and methods described herein utilize the temporal activation arrangement of various structures within the brain to diagnose MDD.

Further, these pathological temporal arrangements provide objective biomarkers for diagnoses of many forms of MDD. Certain treatments described herein can be used to treat MDD and other neuropsychiatric conditions, and when the treatment is efficacious, the pathological temporal activation pattern reverts to a healthy temporal activation pattern. Verification of treatment efficacy can be objectively determined by confirming the shift to healthy temporal arrangement post-stimulation. While the below focuses particularly on L-DLPFC—ACC based MDD, as can be readily appreciated, different pathological brain circuities may be associated with other classes of depression, or other neurological conditions. As such, similar approaches can be used with respect to different brain structures relevant to said other neurological conditions without departing from the scope or spirit of the invention.

In particular, in numerous embodiments, pathological temporal activation patterns can be changed into healthy temporal activation patterns via treatment with iboga alkaloids. Iboga alkaloid treatments are discussed in U.S. patent application Ser. No. 18/467,343, filed Sep. 14, 2023, titled “Methods of Treatment with an Iboga Alkaloid”, the disclosure of which is hereby incorporated by reference in its entirety. Treatment verification systems are discussed in further detail below.

### Treatment Verification Systems

Treatment verification systems obtain functional brain imaging data before and after treatment in order to determine objectively whether or not the treatment has worked. In numerous embodiments, treatment verification systems as described herein can provide an objective indication of whether or not a particular treatment is appropriate for a given patient based on the pre-treatment functional brain imaging data. In many embodiments, neurostimulation targeting systems identify whether or not a given patient has a neurological condition which can be identified via spatiotemporal connectivity analysis, and/or whether or not they are likely to be a responder to various treatments such as, but not limited to, treatment with iboga alkaloids.

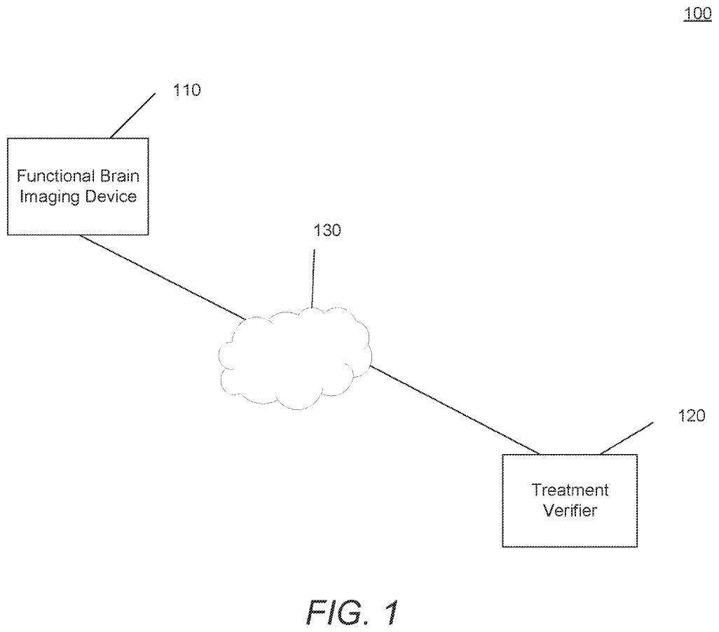

Turning now to FIG. 1, a treatment verification system in accordance with an embodiment of the invention is illustrated. System 100 includes a functional brain imaging device 110. Functional brain imaging devices are capable of imaging a patient's brain and recording a time-series of activation at different locations in the patient's brain. In numerous embodiments, the functional brain imaging device an fMRI machine, but any number of different functional brain imaging modalities can be used as appropriate to the requirements of specific applications of embodiments of the invention.

System 100 further includes a treatment verifier 120. Treatment verifiers receive functional brain imaging data pre-and post-treatment and verify treatment based on spatiotemporal activation patterns within the functional brain imaging data. In many embodiments, the treatment verifier identifies whether or not a patient has a neurological condition marked by abnormal spatiotemporal activation patterns. Treatment verifiers can determine whether or not an individual is likely to be a responder to neurostimulation. In various embodiments, conditions that are not marked by abnormal spatiotemporal activation patterns are unlikely to respond to neurostimulation. However particular abnormal spatiotemporal activation patterns may also indicate a non-responsive patient.

The functional brain imaging device and the treatment verifier communicate over network 130. Networks can be made up of multiple networks or direct connections that are wired or wireless. In various embodiments, components of the system are not directly networked and instead data is passed between components via a physically transferred machine-readable medium. In various embodiments, treatment verifiers operate on their own without the need for direct connection with functional brain imaging devices.

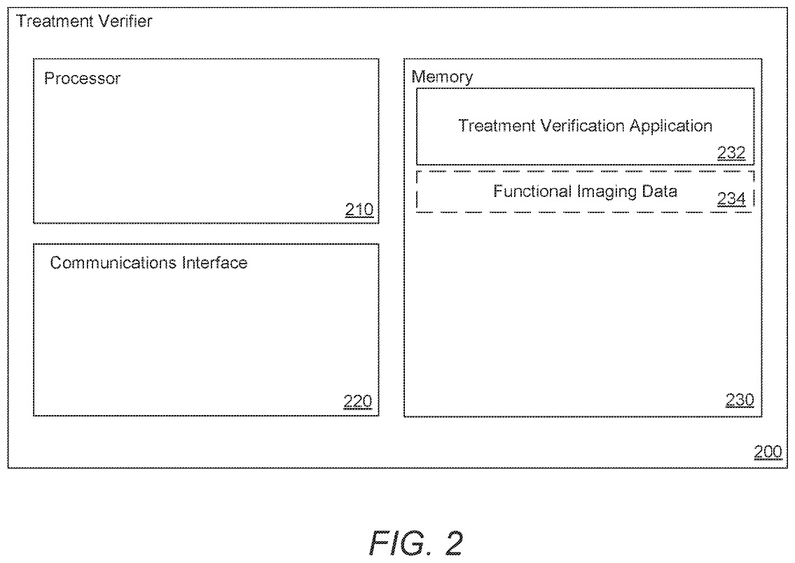

Turning now to FIG. 2, a treatment verifier in accordance with an embodiment of the invention is illustrated. Treatment verifier 200 has a processor 210. Processors can be a central processing unit (CPU), a graphics processing unit (GPU), an application-specific integrated circuit (ASIC), field-programable gate array (FPGA), and/or any other logic circuity as appropriate to the requirements of specific applications of embodiments of the invention. In various embodiments, treatment verifiers have more than one processor.

The treatment verifier 200 further includes an input/output (I/O) interface 220. I/O interfaces can communicate via wired and/or wireless modalities to other devices such as (but not limited to) neurostimulators, functional brain imaging devices, displays, and/or any other electronic device as appropriate to the requirements of specific applications of embodiments of the invention.

Treatment verifier 200 additionally contains a memory 230. Memory 230 can be made of volatile memory, nonvolatile memory, and/or any combination thereof. Memory 230 contains a target generation application 232 and is capable of storing functional imaging data 234. In many embodiments, treatment verification applications configure the processor to use received functional imaging data of a patient's brain to select a treatment protocol based on the patient's specific activation pattern. In various embodiments, treatment verification applications can configure the processor to determine if a patient will be a responder to treatment with iboga alkaloids. While a particular architecture for a treatment verifier is shown, any number of different computing architectures can be used without departing from the scope or spirit of the invention. Processes for treatment verification are discussed in further detail below.

### Treatment Verification

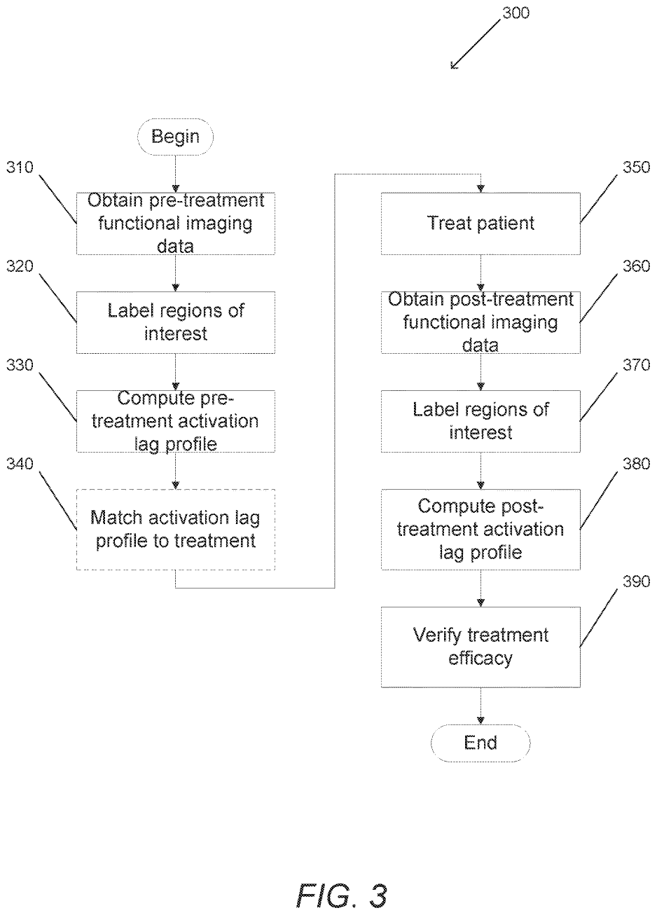

Treatments as contemplated herein are selected based on activation time in correlated brain structures such that treatment is intended to reverse the direction of activation in order to treat a neurological condition. Turning now to FIG. 3, a flow chart for verifying effective treatment using activation time as a biomarker in accordance with an embodiment of the invention is illustrated. Process 300 includes obtaining (310) functional imaging data of a patient's brain prior to treatment. In many embodiments, the functional imaging data is fMRI data containing blood oxygen level dependent (BOLD) signals for different voxels representing different brain regions. However, similar BOLD signals can be obtained using any of a variety of different modalities, and further similar temporal measurement of brain activation can be used as appropriate to the requirements of specific applications of embodiments of the invention.

Regions of interest are labeled (320) such that each voxel is assigned to a particular brain structure. In many embodiments, labeling is achieved using a brain atlas. Based on activation over time between correlated regions of interest, pre-treatment activation lag is computed (330). In many embodiments, the activation lag imputes a direction of signal flow within the brain. In this way, the activation lag is spatiotemporal, reflecting which connected portions of the brain are active at which time relative to each other. In numerous embodiments, the pre-treatment activation lag is matched (340) to a particular treatment such as (but not limited to) treatment using iboga alkaloids previously identified as being effective in reversing pathological temporal activation patterns.

The patient is then treated (350). A post-treatment functional imaging data is obtained (360) in a similar manner as described above. The same regions of interest are labeled (370) in the post-treatment functional imaging data, and a post-treatment activation lag profile is calculated (380). If the pathological temporal activation pattern seen in the pre-treatment activation lag profile is modified and/or reversed in the post-treatment activation lag profile, treatment efficacy is verified (390). As can be readily appreciated, post-treatment functional imaging data can be obtained multiple times at different time points after treatment (or during ongoing treatment) in order to confirm continuing efficacy and/or the point at which treatment has taken effect. Similarly, the specific point at which post-treatment functional imaging data is taken can be modified based on the expected time for the treatment to take effect, which can be anywhere from hours to weeks.

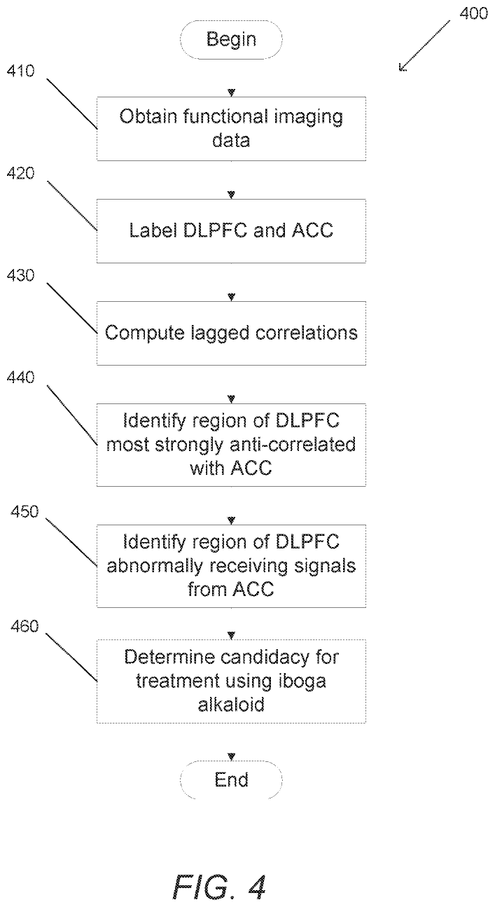

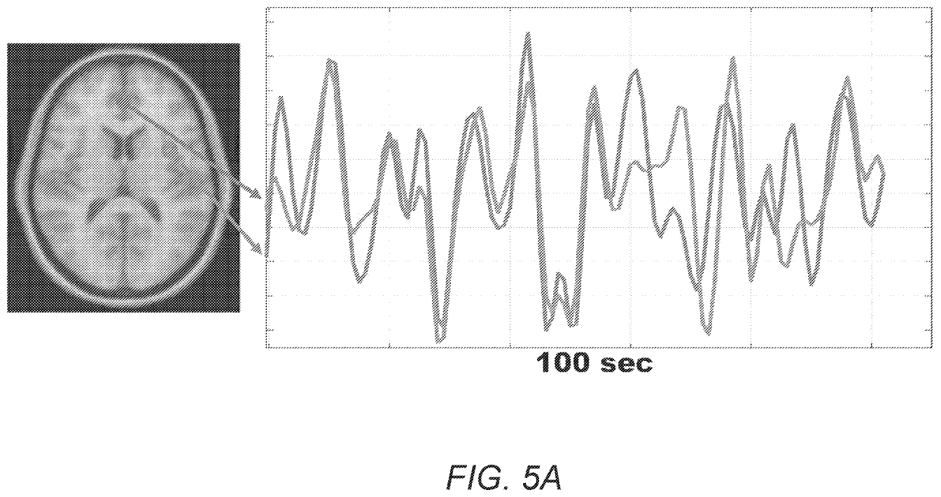

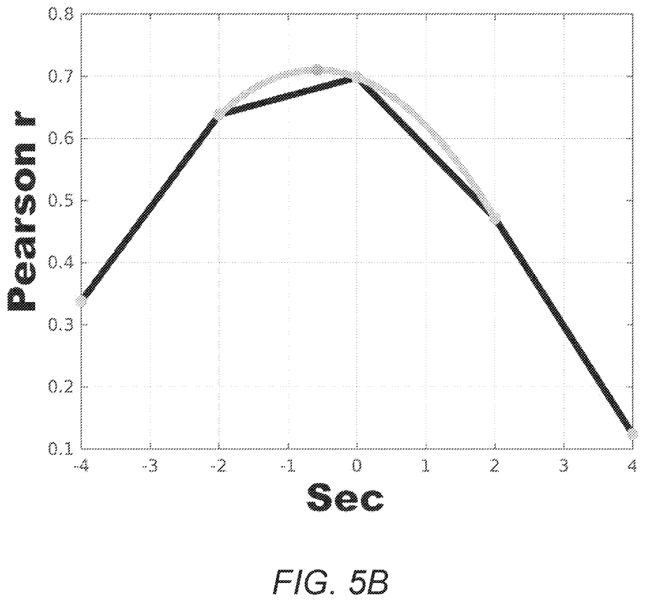

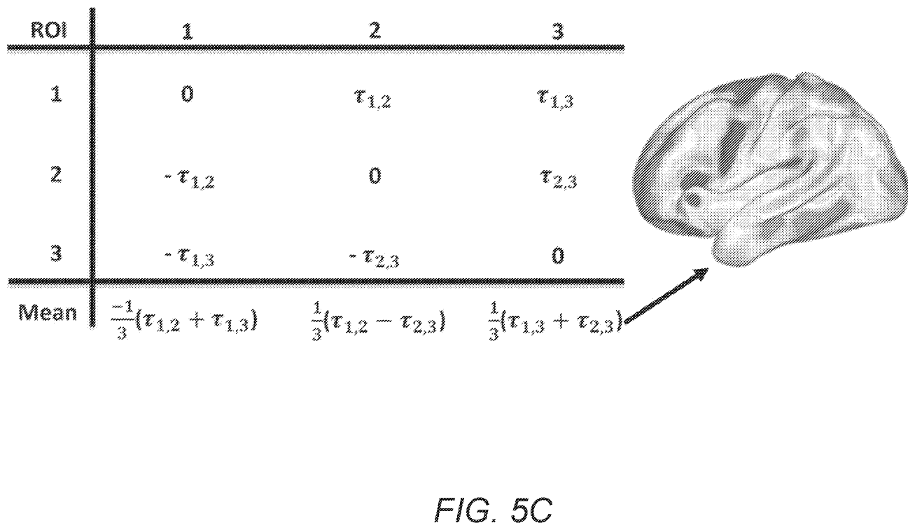

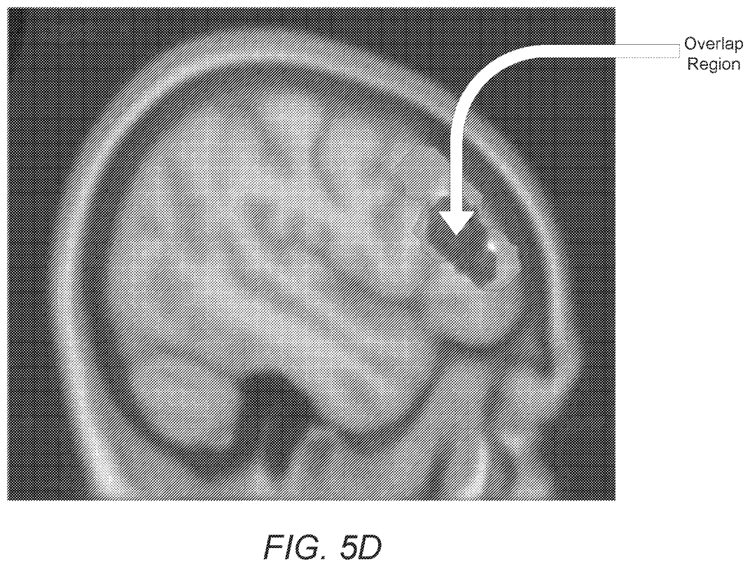

Turning now to FIG. 4, a similar process for verifying iboga alkaloids as a viable pharmaceutical treatment modality for MDD accordance with an embodiment of the invention is illustrated. Process 400 includes obtaining (410) functional imaging data. In many embodiments, the functional imaging data is a resting state fMRI (rs-fMRI). Within the functional imaging data, the dorsolateral prefrontal cortex (DLPFC) and the anterior cingulate cortex (ACC) are labeled (420). In many embodiments, the DLPFC and ACC are identified using segmentation of structural MRI scans, and/or any other structural scan. Using the anatomical cingulate area as a reference region, lagged correlations in the BOLD signal are computed (430) to generate a whole-brain lagged correlation map. An example BOLD signal for the DLPFC and the ACC are illustrated in FIG. 5A. The lagged correlation map includes two main parameters: peak correlation magnitude, and peak temporal delay.

In many embodiments, to calculate lagged correlations, first lags for all pairs of voxels in gray matter in the functional imaging data are computed. An example cross-correlation function for the time series in FIG. 5A is illustrated in FIG. 5B. In the illustrated embodiment, yellow markers and the black line depict empirically measured cross correlation. The green curve depicts a parabolic interpolation about the peak, with the orange marker at the interpolated peak showing a temporal offset of approximately 0.6 sec.

These results are assembled into time-delay (TD) matrices, which have dimensions voxels x voxels and entries in units of seconds. TD matrices represent the lag between all pairs of voxels in gray matter. The mean over all columns of the TD matrix is computed to yield a lag projection map. Lag projection maps topographically represent the mean lag between each voxel and the rest of the brain. The TD matrix format and an example lag projection map are illustrated in FIG. 5C.

The region of the DLPFC most strongly anticorrelated with the ACC is identified (440). In many embodiments, a threshold-based clustering on peak correlation magnitude can be applied to isolate the most strongly anticorrelated region after accounting for signaling delay. The region of the DLPFC which abnormally receives signals from the ACC is identified (450), i.e. the region of the DLPFC which lags the ACC.

The overlap between the two identified DLPFC regions represents the intersection of the peak anti-correlation magnitude with the ACC and the abnormal directed signal receivers in the DLPFC. An example overlapping area of the two identified regions is illustrated in FIG. 5D.

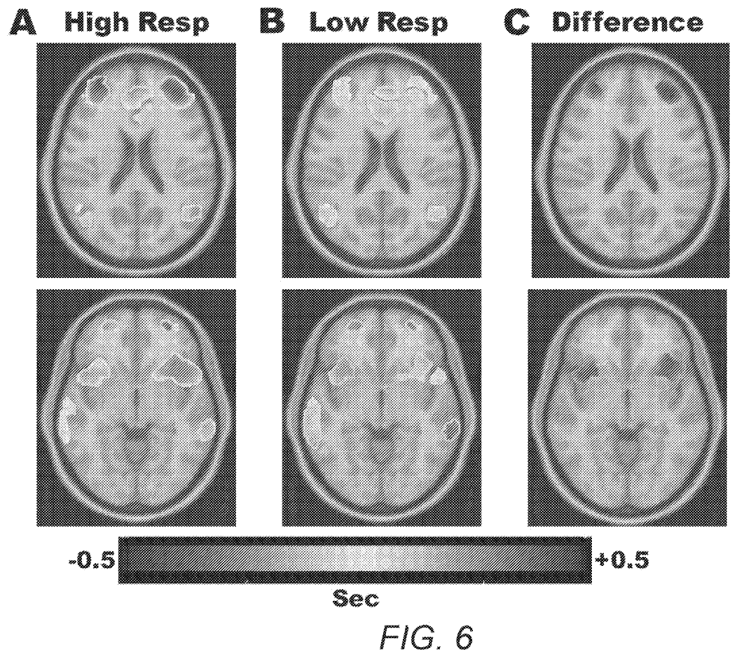

As noted above, the specific regions may be different for different conditions, however similar processing steps can be applied to identify targets for other conditions as appropriate to the requirements of specific applications of embodiments of the invention. Using the identified brain activation, whether or not a patient is a good candidate for treatment using an iboga alkaloid is determined (460). FIG. 6 shows baseline differences in ACC latency structure between high responders and low responders to iboga alkaloid treatment. If similar latency patterns are present in a given patient, it may suggest that they are less likely to respond, or may respond to a lesser degree than would otherwise be expected.

Although systems and methods for treatment verification are discussed above, many different architectures and methods can be implemented in accordance with many different embodiments of the invention which utilize spatiotemporal connectivity in a similar manner. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A treatment verification system, comprising:
a processor; and
a memory containing a treatment verification application that configures the processor to:
obtain a pre-treatment functional imaging data of a patient's brain;
identify pre-treatment disordered lagged correlations between different brain regions associated with a neuropsychiatric disorder using the functional imaging data;
treat the patient with an iboga alkaloid or salt thereof in an amount to achieve a physiologic effect;
obtain a post-treatment functional imaging data of the patient's brain;
identify reversion of the pre-treatment disordered lagged correlations in the post-treatment functional imaging data; and
provide a verification that treatment with the iboga alkaloid or salt thereof provided a therapeutic effect in treating the neuropsychiatric condition.

2. The treatment verification system of claim 1, wherein the neuropsychiatric condition is major depressive disorder.

3. The treatment verification system of claim 1, wherein the neuropsychiatric condition is suicidal ideation.

4. The treatment verification system of claim 1, wherein the iboga alkaloid or salt thereof is provided with a cardioprotective agent.

5. The treatment verification system of claim 1, wherein to identify pre-treatment disordered lagged correlation, the treatment verification application further configures the processor to:
label voxels in the pre-treatment functional brain imaging data that make up a dorsolateral prefrontal cortex and an anterior cingulate cortex in the pre-treatment functional brain imaging data using a brain atlas;
generate a whole-brain lagged correlation map using the voxels labelled as making up the anterior cingulate cortex as a reference region, where the whole-brain lagged correlation map comprises a peak correlation magnitude and a peak temporal delay;
compute a lag for all pairs of voxels containing grey matter; and
assemble the computed lags into a time-delay matrix.

6. The treatment verification system of claim 5, wherein to identify pre-treatment disordered lagged correlations, the treatment verification application further configures the processor to compute the mean delay over all columns of the time-delay matrix to produce a lag projection map.

7. The treatment verification system of claim 1, wherein to identify post-treatment disordered lagged correlation, the treatment verification application further configures the processor to:
label voxels in the post-treatment functional brain imaging data that make up a dorsolateral prefrontal cortex and an anterior cingulate cortex in the pre-treatment functional brain imaging data using a brain atlas;
generate a whole-brain lagged correlation map using the voxels labelled as making up the anterior cingulate cortex as a reference region, where the whole-brain lagged correlation map comprises a peak correlation magnitude and a peak temporal delay;
compute a lag for all pairs of voxels containing grey matter; and
assemble the computed lags into a time-delay matrix.

8. The treatment verification system of claim 7, wherein to identify post-treatment disordered lagged correlations, the treatment verification application further configures the processor to compute the mean delay over all columns of the time-delay matrix to produce a lag projection map.

9. The treatment verification system of claim 1, wherein pre-treatment functional imaging data and post-treatment functional imaging data are produced using a magnetic resonance imaging machine.

10. The treatment verification system of claim 1, wherein the post-treatment functional imaging data is obtained between 1 day and 1 month post-treatment.

11. A method of verifying treatment with iboga alkaloids, comprising:
obtaining a pre-treatment functional imaging data of a patient's brain;
identifying pre-treatment disordered lagged correlations between different brain regions associated with a neuropsychiatric disorder using the functional imaging data;
treating the patient with an iboga alkaloid or salt thereof in an amount to achieve a physiologic effect;
obtaining a post-treatment functional imaging data of the patient's brain;
identifying reversion of the pre-treatment disordered lagged correlations in the post-treatment functional imaging data; and
providing a verification that treatment with the iboga alkaloid or salt thereof provided a therapeutic effect in treating the neuropsychiatric condition.

12. The method of verifying treatment with iboga alkaloids of claim 11, wherein the neuropsychiatric condition is major depressive disorder.

13. The method of verifying treatment with iboga alkaloids of claim 11, wherein the neuropsychiatric condition is suicidal ideation.

14. The method of verifying treatment with iboga alkaloids of claim 11, wherein the iboga alkaloid or salt thereof is provided with a cardioprotective agent.

15. The method of verifying treatment with iboga alkaloids of claim 11, wherein identifying pre-treatment disordered lagged correlation comprises:
labeling voxels in the pre-treatment functional brain imaging data that make up a dorsolateral prefrontal cortex and an anterior cingulate cortex in the pre-treatment functional brain imaging data using a brain atlas;
generating a whole-brain lagged correlation map using the voxels labelled as making up the anterior cingulate cortex as a reference region, where the whole-brain lagged correlation map comprises a peak correlation magnitude and a peak temporal delay;
computing a lag for all pairs of voxels containing grey matter; and
assembling the computed lags into a time-delay matrix.

16. The method of verifying treatment with iboga alkaloids of claim 15, wherein to identify pre-treatment disordered lagged correlations, the treatment verification application further configures the processor to compute the mean delay over all columns of the time-delay matrix to produce a lag projection map.

17. The method of verifying treatment with iboga alkaloids of claim 11, wherein identifying post-treatment disordered lagged correlation comprises:
labeling voxels in the post-treatment functional brain imaging data that make up a dorsolateral prefrontal cortex and an anterior cingulate cortex in the pre-treatment functional brain imaging data using a brain atlas;
generating a whole-brain lagged correlation map using the voxels labelled as making up the anterior cingulate cortex as a reference region, where the whole-brain lagged correlation map comprises a peak correlation magnitude and a peak temporal delay;
computing a lag for all pairs of voxels containing grey matter; and
assembling the computed lags into a time-delay matrix.

18. The method of verifying treatment with iboga alkaloids of claim 17, wherein identifying post-treatment disordered lagged correlations further comprises computing the mean delay over all columns of the time-delay matrix to produce a lag projection map.

19. The method of verifying treatment with iboga alkaloids of claim 11, wherein pre-treatment functional imaging data and post-treatment functional imaging data are produced using a magnetic resonance imaging machine.

20. The method of verifying treatment with iboga alkaloids of claim 11, wherein the post-treatment functional imaging data is obtained between 1 day and 1 month post-treatment.
